(19) United States
(12) Patent Application Publication
(10) Pub. No.: US 2008/0057138 A1
(43) Pub. Date: Mar. 6, 2008

(54) RESTORATIVE SKIN CREAM

(75) Inventors: Gordon Owen Telford, Victoria (CA); Emma Rozenberg, Coquitlam (CA)

Correspondence Address: OYEN, WIGGS, GREEN & MUTALA LLP;480 - THE STATION, 601 WEST CORDOVA STREET, VANCOUVER, BC, V6B 1G1, US

(73) Assignee: Telford Holdings Ltd., Victoria (CA)

(21) Appl. No.: 11/470,339

(22) Filed: Sep. 6, 2006

(51) Int. Cl. A61K 31/34; A61K 31/355; A61K 31/07; A61K 31/203; A61K 31/375

(52) U.S. Cl. 424/642; 514/559; 514/474; 514/458; 514/725

(57) ABSTRACT

This invention relates to topical creams which retard sun exposure damage to skin and restore skin health. More particularly, this invention pertains to a novel topical skin cream formulation which retards sun damage to skin and restores skin health comprising: (a) vitamin A retinyl at a concentration of 0.2% to 10% weight, and/or retinol at a concentration of 0.2% to 10% weight; (b) vitamin C at a concentration of 5 % to 30% weight; (c) vitamin E at a concentration of 1% to 10% weight; (d) zinc sulfate at a concentration of 2% to 5% weight; and (e) the remainder, water and skin compatible thickeners, emulsifiers, emollients and moisturizers, having a pH of about 3 to 6.

### FIELD OF THE INVENTION

This invention relates to topical creams which restore and maintain skin health. More particularly, this invention pertains to novel topical skin cream formulations and methods of applying the formulations which retard sun damage to skin and restore and maintain skin health.

### BACKGROUND OF THE INVENTION

Dermatologists believe that 80 percent of the visible signs of aging, that is, dullness, altered pigmentation, lines (wrinkles) and laxity of the skin are due to prolonged sun exposure, beginning from as early as a person's childhood. Continued sun exposure furthers skin damage. The consequences of continued sun exposure are photo aging, actinic keratoses and malignancies and immunosuppression.

A number of patents have issued over the years relating to compositions that purportedly restore the health of skin that has suffered sun exposure damage.

U.S. Pat. No. 6,753,449, Pinnell et al., discloses an aqueous topical composition comprising olive leaf extract, vitamin C (15-20%), vitamin A (1%), α-tocopherol (1-2%) and zinc sulfate (1-5%). The carrier is an aqueous formulation comprising water, an alcohol and a surfactant. The composition is claimed to have antioxidant effects and to protect against sunburn.

U.S. Pat. No. 4,704,280, Bates, discloses an aqueous cosmetic lotion comprising aloe vera, vitamin C (0.1-7%), vitamin A (0.005% -0.1%), vitamin E (0.1%-3.1%) and a “therapeutic compound” of zinc (0.03%-0.1%). The aqueous carrier is water. The Bates lotion is claimed to have skin-softening effects.

U.S. Pat. No. 5,378,461, Neigut, discloses a balm comprising coenzyme Q, vitamin A (8%) and vitamin E (16-19%). This patent cites a source of coenzyme Q containing an unspecified amount of vitamin C and zinc in its formulation. The balm can be a cream. The oil-based carrier may be a squalene or olive oil. The carrier also includes a surfactant to maintain uniform consistency between the oil and water soluble components. The cream is claimed to treat skin damage caused by toxins, radiation and aging.

U.S. Pat. No. 4,372,296, Fahim, discloses a topical composition comprising zinc sulfate (1-4%), vitamin C (2-6%) and undisclosed amounts of vitamins E and A. The carrier may be a cream comprising a mixture of two different alcohols, petrolatum, mineral oil and propylene glycol. The composition may be used in the treatment of acne and other skin disorders.

WO 95/26183, Vuorela et al., discloses a composition comprising wheat shoot extract (2.5%), thyme oil, vitamin A (0.1-0.2%), a-tocopherol acetate (0.5-1.5%) and zinc sulfate (0.2-0.6%). The wheat shoot extract is said to contain a “significant concentration” of vitamin C. The composition can be a cream (water/oil or oil/water emulsion ointment) and purportedly rejuvenates skin, cures sunburn and treats skin disorders.

U.S. Pat. No. 5,554,647, Perricone, discloses a topical composition comprising an acetylcholine precursor, vitamin C (8-10%), vitamin E (1-3%) and zinc (0.5-1.5%). The “dermatologically acceptable carrier” may contain water, an alcohol and oils. The composition is claimed to treat and prevent skin damage and retard skin aging.

U.S. Pat. No. 5,750,124, Gohla et al., discloses a skin care emulsion comprising a three phase water/oil/water emulsion. The emulsion may include unspecified concentrations of antioxidants, including vitamin C, vitamin A, vitamin E and zinc sulfate.

U.S. Application No. 2003/0049214, Muller, U.S. Application No. 2004/0037795, Hamer et al., U.S. Pat. Nos. 6,793,929 and 6,649,577, Bleckman et al., U.S. Pat. No. 6,585,983 and U.S. Application No. 2003/0175221, Gers-Barlag et al., and U.S. Pat. No. 6,503,518, von der Fecht et al., all disclose topical compositions that may include vitamin C, vitamin A, vitamin E and zinc sulfate in undisclosed proportions. The disclosed concentration ranges of these antioxidants is 0.001% to 30%, and in particular for vitamins E and A, preferably between 0.001% and 10%. The compositions may be a cream (typically an oil/water or water/oil emulsion).

WO 2004/006869, Hauser et al., discloses an absorbent applicator which may be impregnated with a lipid phase containing tocopherols, vitamin C and zinc sulfate. U.S. Application No. 2004/0102429, Shanta et al., discloses an anti-in-itant cream composition comprising an organic zinc salt (0.1%-0.2%), and undisclosed amounts of vitamins C, A and E. U.S. Application No. 2003/0232091, Shefer et al., discloses a cream containing stabilized retinol for treating and preventing skin disorders and photo aging. The cream may include unspecified amounts of vitamins C and E.

U.S. Pat. No. 4,938,969, Meisner et al., discloses a skin wrinkle reduction cream comprising zinc sulfate (0.5-5%) and vitamin C (2-20%). U.S. Pat. No. 6,217,914, Meisner, discloses a topical cream for protecting the skin against UV radiation. The cream preferably comprises 15-25% vitamin C and 0.5-5% zinc sulfate.

U.S. Pat. No. 6,440,465, Meisner, discloses compositions and methods of treatment for psoriasis and related skin ailments. The compositions include topical skin formulations of glucosaine in an emollient base such as moisturizing cream. In addition to glucosamine, the formulations may include keratolytic substances such as coal tar extract or salicylic acid. The formulations may also include glucosamine and antioxidant anti-inflammatoly herbal extracts such as oleuropein and berberine in an emollient base. U.S. Pat. No. 6,444,699, Meisner, discloses a topical cream comprising a zinc salt and at least 5% vitamin C for the treatment of aging and photodamage of skin.

U.S. Pat. No. 5,140,043, Pinnell et al., discloses an aqueous vitamin C stabilizing composition comprising 3-20% vitamin C in water. U.S. Pat. No. 6,524,599, Pinnell, discloses a skin care composition comprising milk thistle extract, α-tocopherol (1-5%), water and an unspecified amount of vitamin A. The composition is claimed to function by “delaying, minimizing, preventing and/or ameliorating the signs of skin aging”.

The foregoing examples of the related art and limitations related thereto are intended to be illustrative and not exclusive.

### SUMMARY OF THE INVENTION

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

The invention is directed to a formulation for topical application to the skin of a human being comprising: (a) vitamin A, and complexes thereof, at a concentration of 0.2% to 10% weight and/or retinol at a concentration of 0.2% to 10% by weight; (b) vitamin C, and salts and complexes thereof, at a concentration of 5% to 30% weight; (c) vitamin E, and complexes thereof, at a concentration of 1% to 10% weight; (d) zinc sulfate at a concentration of 2% to 5% weight; and (e) and the remainder, water and skin compatible thickeners, emulsifiers, emollients and moisturizers, to form a cream that has a pH of about 3 to 6.

The vitamin A can be vitamin A palmitate and/or retinol. The vitamin C can be L-ascorbic acid. The vitamin E can be α-tocopherol, β-tocopherol, γ-tocopherol or δ-tocopherol. α-tocopherol is preferred.

The invention in a particular embodiment is directed to a formulation for topical application to the skin of a human being comprising: (a) vitamin A palmitate or retinyl palmitate at a concentration of 0.2% to 10% weight and/or retinol at a concentration of 0.2% to 10% weight; (b) L-ascorbic acid at a concentration of 5% to 30% weight; (c) α-tocopherol at a concentration of 1% to 10% weight; (d) zinc sulfate at a concentration of 2% to 5% weight; and (e) the remainder, water and skin compatible thickeners, emulsifiers, emollients and moisturizers, to form a cream that has a pH of about 3 to 6.

The formulation can comprise two components which are topically applied to the skin separately and, if desired, at different times: (a) L-ascorbic acid and α-tocopherol at a pH of 3.5; and (b) vitamin A palmitate and/or retinyl palmitate, α-tocopherol and zinc sulfate at a pH of 5.5.

In the topical formulation, in one embodiment, the vitamin A palmitate or retinyl palmitate can have a concentration of 0.2% to 10% weight, the retinol can have a concentration of 0.2% to 10% weight, the L-ascorbic acid can have a concentration of 5% to 30% weight, the α-tocopherol can have a concentration of 1% to 10% weight, and the zinc sulfate can have a concentration of 2% to 5% weight. The remainder of the formulation for one cream can be water and hydrogenated polydecene, PEG-30 dipolyhydroxystearate, glycerin, ethoxylated propylene oxide copolymer, sodium hydroxide, magnesium sulfate, phenoxyethanol, butylated hydroxytoluene, xanthan gum, butyl paraben, methyl paraben, ethyl paraben, and propylparaben, to form a cream that has a pH of about 3 to 6. The remainder of the formulation for a second cream can be water and emulsifying wax NF, caprylic/capric triglyceride, pentylene glycol, bisabolol, panthenol, stearic acid, butyl methoxydibeizoylmethane, phenoxyethanol, butylated hydroxytoluene, tetrasodium ethylenediamimine tetraacetate, butyl paraben, methyl paraben, ethyl paraben, and propylparaben, to form a cream that has a pH of about 3 to 6.

The invention in a further embodiment is directed to a formulation for topical application to the skin of a human being comprising: (a) vitamin A palmitate or retinyl palmitate at a concentration of 1% weight and/or retinol at a concentration of about 1% weight; (b) L-ascorbic acid at a concentration of about 20% weight; (c) α-tocopherol at a concentration of about 2.5% weight; (d) zinc sulfate at a concentration of about 3.5% weight; and (e) the remainder, water and skin compatible thickeners, emulsifiers, emollients and moisturizers, to form a cream that has a pH of about 3 to 6. The formulations can also include lipids, antioxidants, anti-irritants, humectants, preservatives and chelating agents.

One of the formulations can be at least a two-phase emulsion formulation comprising an aqueous phase and a non-aqueous phase and the zinc sulfate can be in the aqueous phase and the vitamin A palmitate or retinyl palmitate and/or the retinyl palmitate and the α-tocopherol can be in the non-aqueous phase. The formulation can also include a UV absorbing agent such as butyl methoxydibenzolymethane or octyl methoxycinnamate.

The invention also relates to a multiple emulsion of the water/oil/water (W/O/W) type comprising a continuous external aqueous phase, an oily phase dispersed therein and a second (internal) aqueous phase dispersed in the oily phase. The three-phase formulation for topical application to the skin of a human being can comprise: (a) a water-based phase containing vitamin C at a concentration of about 5% to 30% weight; (b) an oil-based phase containing α-tocopherol at a concentration of about 1% to 10% weight; (c) an external and an internal aqueous phase containing physiologically tolerated organic and/or inorganic electrolytes with mono-, di-, or trivalent cations at a concentration of about 0.3% to 5% by weight based on the total weight of the formulation; (d) other auxiliaries, additives and/or active compounds customary in cosmetics or medical compositions can be incorporated into the oily phase and/or the aqueous phase; (e) further auxiliaries and/or additives can be incorporated into the oily phase and/or the aqueous phases to stabilize multiple emulsion droplets.

The invention also pertains to a method of topically treating human skin to retard damage caused by exposure to sunlight comprising applying to the skin during the day a cream comprising L-ascorbic acid, α-tocopherol, water and physiologically tolerated auxiliaries, additives and/or active compounds, at a pH of 3.5; and in the evening applying to the skin a cream comprising vitamin A palmitate, α-tocopherol, zinc sulfate, water, lipids, antioxidants, anti-irritants, humectants, preservatives and chelating agents at a pH of 5.5.

The invention also pertains to a method of topically treating human skin to retard damage caused by exposure to sunlight which consists of applying to the skin during the day a multiple emulsion cream having at least two aqueous phases and at least one non-aqueous phase comprising L-ascorbic acid in the aqueous phase at a pH of 3.5 and α-tocopherol in the non-aqueous phase. The remainder of the formulation for the day cream can be water and hydrogenated polydecene, PEG-30 dipolyhydroxystearate, glycerin, ethoxylated propylene oxide copolymer, sodium hydroxide, magnesium sulfate, phenoxyethanol, butylated hydroxytoluene, xanthan gum, butyl paraben, methyl paraben, ethyl paraben, and propylparaben.

The night cream formulation can have at least a two-phase emulsion comprising zinc sulfate in the aqueous phase and vitamin A palmitate or retinyl palmitate and α-tocopherol in the non-aqueous phase at a pH of 5.5. The remainder of the formulation for the night cream can be water and emulsifying wax NF, caprylic/capric triglyceride, pentylene glycol, bisabolol, panthenol, stearic acid, butyl, methoxydibenzoylmethane, phenoxyethanol, butylated hydroxytoluene, tetrasodium ethylenediamimine tetraacetate, butyl paraben, methyl paraben, ethyl paraben, and propylparaben.

### DETAILED DESCRIPTION OF THE INVENTION

Throughout the following description specific details are set forth in order to provide a more thorough understanding of the invention to persons skilled in the art. However, well known elements may not have been shown or described in detail to avoid unnecessarily obscuring the disclosure. Accordingly, the description is to be regarded in an illustrative, rather than a restrictive, sense.

### Photo Aging

Photo aging in Fitzpatrick skin types 1-3 occurs in a series of logical and sequential diagnostic steps. The most important constant is photo distribution. The earliest signs are persistent pigmentary changes. The first diagnostic sign is actinic lentigo (freckles which persist through winter) which are present initially on the hands and face. Persons with blond hair before the age of 10 are more susceptible but not exclusive to this damage, often exhibiting in their 30's diagnostic progressive skin changes. These include persistent redness and hyperpigmentation of the neck and frontal V of the neck and scattered hypomelanotic patches. The skin in these three types loses its natural sheen and appears dull which reflects epidermal dysplasea.

The second diagnostic change is the appearance of fine lines (wrinkles) which persist after 20 seconds at rest. These are initially presented in the periorbital and lower lip surface and progressively become more numerous and widespread. Deep lines (exaggerated folds) occur first in the periorbital area (crows feet) followed by the upper vermillion border (lipstick bleed) and progressively become longer, deeper and more widespread, i.e. posterior cheeks (California raisin).

The third stage, skin laxity, is first noticed as a loosening of the skin which progresses to more overt signs of “bags” under the eyes; “jowls” on the jaw line and loosening of the neck. Specific individual diagnostic signs include elastosis (yellow dermal papules) on the forehead (solar elastosis), and at the back of the neck (Elastosis nuchae), actinic acne (Favre Rachochot), milia, and senile purpura.

### Actinic Keratoses and Malignancies

In the predisposed person, chronic sun exposure results in actinic keratoses and non-melanoma skin cancers (basal cell, squamous cell, Bowen's disease and keratoacanthomas). Collectively, these cancers account for over 50% of all new cancers that occur annually. These cancers typically occur with increasing frequency after 55 years of age and are expected to markedly increase in absolute numbers as the human population ages. There is a clear dose response relationship between intensity and duration of sun exposure in the frequency of squamous cell carcinoma that occurs in humans. Additionally, there is a strong but not necessarily direct relationship to other non-melanoma skin cancers and melanoma. Clearly, there is an unidentified confounding variable which can probably be attributed to photo-induced immunosuppression.

### Photoinduced Immunosuppression

The putative solar wavelengths for photo-induced immunosuppession are in the ultraviolet spectrum (UVA and UVB). UVB is the wavelength responsible for sunburns. An under-appreciated observation is described by Streilein. He noted that UVB induced immunosuppression occurred in low dose exposure in ⅓ of Caucasian subjects and that this characterized 95% of patients with two or more skin cancers. He further noted that the same reaction pattern, designated UVB susceptibility, was present in 100% of melanoma patients. He hypothesized that this UVB susceptibility is the predisposing factor in the development of clinically overt malignancies.

UVA is the predominant wavelength in and component of sun exposure. UVA is not seasonally affected and is not blocked effectively by commercial sunscreens. Results from recent micro dissection studies in squamous cell carcinoma and actinic keratoses suggest that UVA is the major carcinogen in the basal layer whereas UVB exerts its effects in the supra basal layers.

### The Use and Limitations of Sunscreens

Sunscreens have been widely available to the general public for at least thirty years. Sunscreens are the established commercial agent for photo protection. Compliance rates are an ongoing problem. Exit interviews from beach goers in Texas on a hot summer day revealed that 78% of women and 34% of males used sunscreens; only 41% treated all exposed areas and 73% experienced sunburns. Common reasons for sunscreen failure included inadequate thickness of application of the sunscreen on the skin and failure to reapply the sunscreen after swimming. Up to 80% of UV exposure occurs as incidental exposure which is blocked only if sunscreens are applied everyday. Traditional sunscreens function in the upper layers of the epidermis and work by reflecting, dispersing or absorbing incident light. Historically, the focus of sunscreen development has been to provide UVB protection. Current research points out that UVA is the major component of ultraviolet light and that none of the currently available sunscreens offer full protection in the UVA range. Use of sunscreen combined with multiple antioxidants may offer protection to the skin in the form of absorption of free radicals in both the UVA and UVB ranges and improve the persistence of sunscreen effect for up to 4 days after application. The subject invention is an extension of previous biological sunscreens which have an SPF of 6. This formulation results in an SPF of 26. The formulation's SPF resulting from a direct cellular effect is additive to standard sunscreens.

### Free Radicals Theory

Sun damage occurs as a result of a chemical interaction between the sun's energy (photons) and cutaneous structure of the person involved. This chemical interaction between the sun and the skin releases free radicals in the skin which progressively damage healthy cells by altering their physical structure.

As Streilein pointed out, one in three people, when exposed to normal sunlight, develops a unique form of immunosuppression. This is characteristic of the 95% of people who have multiple skin cancers. A depressed immune system does not recognize premalignancies and allows them to grow. Topical application of a concentrated strength of vitamin A, C, E and zinc sulfate to the skin will prevent immunosuppression from occuring.

The wavelengths associated with free radical damage are in the UV spectrum, UVA and UVB. Free radical production is thought to be the mechanism underlying:
(a) photo-induced pigmentation;
(b) collagen destruction by increasing levels of proteolytic enzymes which dissolve collagens (skin types 1, 3 and 7); and by suppressing new collagen production;
(c) photo-induced immunosuppression; and
(d) skin cancers including melanoma.

It is well established that in the UV range of 290-320, an additional mechanism of direct chromophore absorption induces specific DNA change, which is detrimental.

In addition to sunlight exposure, free radicals are produced by outside agents such as nicotine (identified as being a causative factor in the development of deep wrinkles) and other pollutants found in the air and the environment.

### Antioxidant Absorption of Free Radicals

The human body naturally manufactures certain antioxidants (melanin, ubiquinone, etc.) which protect the skin by absorbing and thereby neutralizing free radicals. There are three major cutaneous exogenous antioxidants, namely, three vitamin molecules A,C, and E. Vitamin C is the major aqueous phase free radical absorber, vitamin E is a major epidermal free radical absorber. Vitamin A is active within the cell nucleus. The free radical and antioxidant interaction with these three vitamins results in neutralization of the destructive capacity of the free radicals and thereby protects vital cellular processes. A deleterious effect of sun exposure is that stores of vitamins in the skin are reduced and these stores are slow to replenish. Further free radical production from sun exposure on the skin then proceeds virtually unopposed.

For each of the three named vitamins, specific parameters have been defined for maximizing physiological effect and percutaneous absorption. The most critical of these can be summarized as:
(1) Vitamin A (in particular, vitamin A palmitate or retinol) which has the ability to stimulate the retinoid receptor at strengths (in non-tretinoin molecules) of 0.2% to 10% weight;
(2) Vitamin C at pH 3.5 or less; particularly the L form, in non-esterified strengths of 5% to 30% weight; and
(3) Vitamin E; particularly α-tocopherol, in strengths of 1% to 10% weight.

### Studies with Topical Antioxidants

### Immediate Enhanced Photoprotection

Each of the three vitamins A, C and E, and complexes thereof, when topically applied individually at therapeutic levels, markedly increases the ability of the skin to absorb free radicals. Each vitamin molecule, as a measurement of this effect, adds the ability to absorb 2 MEDs (minimal erythema dose) of energy or a total of 6 MEDS if all three vitamins are present simultaneously. Topical application of vitamin A palmitate has demonstrated an SPF equivalent to 20. Topical application of retinol has demonstrated an SPF of 20. SPF (Sun Protection Factor) is a recognized measure of ultraviolet screen ability. Simultaneous application of a tri-vitamin regimen of vitamins A, C and E can result in an SPF equivalent of 24. Use of the three vitamins together in effective amounts results in enhanced protection of the skin against sunburn and, unlike sunscreens per se, persists in effect until the molecules are consumed by the body. Persistence has been demonstrated for vitamin C for at least 4 days following a single application. The enhancement of photo protection by the tri-vitamin therapy according to the invention is particularly important as a solar spectrum (UVA, UVB) sun protectant. The effects of simultaneous use of the tri-vitamin regime and sunscreens are additive.

### Prevention of Photo Aging

Each of vitamins A, C and E has been proven to prevent photo aging and photo-induced pigmentation in animal studies. Human trials substantiate this effect for tretinoin (vitamin A) and are limited for C and E and are believed to be non-existent for the tri-vitamin combination of vitamins A, C and E. This effect occurs in both natural aging and photo aging. Individually, each of the three vitamins has been shown to prevent UV induced increases in collagenolytic enzymes and to reverse the inhibition of collagen synthesis.

Relevant literature demonstrates the ability of topical tretinoin (all-trans retinoic acid), isotretinoin, tazarotene and adapalene to reverse some of the signs of chronic photo aging. Particularly these relate to a significant reduction in actinic lentigo, improvement in skin surface texture and reduction in fine lines. Similar changes are observed for topical vitamin C (L-ascorbic acid 5%), which result in the reversal of deeper lines. It is believed that no scientifically acceptable human trials are reported for topical vitamin E.

### Treatment of Photo Aging

The benefits of topical application of tretinoin in the reversal of both intrinsic and photo aging for periods up to 24 months have been demonstrated. These benefits include clinically significant improvement in surface texture, reduction of actinic lentigo and reversal of fine lines, particularly in the periorbital area.

The effects of ultraviolet light exposure on the skin are acutely, sunburn and tanning, and in the long term, photo aging, actinic keratoses, malignancies, and immunosuppression. Sun worshiping lifestyles, geographic location and thinning of the protective ozone layer have led to an epidemic of sun-induced skin disease. In addition to melanin, and other endogenous molecules, the human body relies on exogenous vitamins to protect itself from ultraviolet injury. These are vitamins A, C, and E. Sun exposure depletes skin reserves of these three vitamins, and once depleted, the skin is more prone to skin disease. Acute and chronic sun damage ultimately results. A rate limiting step in the biological process is the inability of the skin to transport enough nutrient vitamin from other areas to replenish its stores in the skin.

### Prevention of Immunosuppression

Each of the antioxidant vitamins A, C and E, has been demonstrated independently to reduce photo-induced immunosuppression when administered both topically and systemically in animal models.

### Prevention of Skin Carcinoma Prevention

Each of the vitamins A, C and E has been demonstrated independently to markedly reduce photocarcinogenesis in animal model studies. Each of the vitamins A, C and E has specific structural and absorption characteristics that allow penetration through the epidermis and subsequently the dermis. Human trials using topical tretinoin (all-trans retinoic acid) substantiate this effect on actinic keratoses. Evidently, no human trials have been undertaken to demonstrate this effect with Vitamin C and E, in combination. It is believed no human trials have been conducted for the tri-vitamin combination of vitamins A, C, and E.

### Vitamin A

The term vitamin A per se is a broad, comprehensive and imprecise term, which includes any molecule capable of inducing specific messenger transport molecules to bring about active metabolic processes. In this discussion, when vitamin A is referred to, it is understood that conceivable, viable, therapeutically effective forms of vitamin A are included in the scope of the invention. Vitamin A palmitate or retinyl palmitate represent stable storage forms for vitamin A which occurs in maximum concentrations physiologically. Vitamin A palmitate is inherently unstable and requires complex formulation technology to maintain its stability. We have found an effective concentration is 0.2% to 10% weight.

### Vitamin C

For effective absorption and demonstrable physiological effect, the characteristics of vitamin C are such that it must be presented in an acidic environment (a pH of 3.5 or less) and for best results should be the pure (L) form of the vitamin C molecule. The addition of stabilized molecules, such as acetates, esters, etc. inhibit absorption. We have found that an effective concentration to produce maximum physiological effect occurs in the range of 5% to 30% weight. The L isomer of topical vitamin C (L-ascorbic acid) is physiologically active. The D isomer of ascorbic acid is not active, and may in fact reduce the effectiveness of the L isomer of vitamin C by competitive inhibition.

### Vitamin E

Vitamin E is a broad term which describes various forms of tocopherols and tocotrienols. They are inherently unstable molecule. In natural sources, vitamin E typically occurs as a series of approximately 8 different isomeric molecules. One, α-tocopherol, has been identified as being particularly physiologically active. Simultaneous presentation of multiple isomers of vitamin E may reduce the effectiveness of α-tocopherol by competitive inhibition. Esterification or altering the structure of α-tocopherol in typical treatment, significantly reduces its efficacy and absorption characteristics. However, the various forms of vitamin E are included within the scope of this invention. We have found that an effective concentration of topical α-tocopherol falls in the range of 1 to 10% weight. The use of vitamin E at concentrations greater than 5% as a topical applicant may be associated with significant comedogenicity.

### Zinc Sulfate

Zinc sulfate has been shown to be an essential co-factor in collagen production. We have found that an effective concentration range is 2% to 5% weight.

Given the unique structures and requirements of each of the three vitamin molecules, and zinc sulfate, and the relatively high but different concentrations of application that are required for efficacy of the invention, the inventors had to overcome a number of formulary problems.

### Formulations

The inventors have developed topical application formulations wherein the three vitamins A, C and E and zinc sulfate are incorporated at the preferred concentration ranges. In one specific embodiment, the vitamins can be applied in two daily stages (tri-vitamin antioxidant therapy of vitamins A, C and E in stable formulation). When these formulations are applied in therapeutic concentrations to the skin surface of humans, they retard changes such as photo aging, skin carcinomas and immunosuppression.

In one aspect, we have developed a two-stage application formulation and have demonstrated the following:
(a) Each of the molecules that comprise the two-stage restorative skin cream, namely vitamins C and E in a first formulation, and vitamins A and E and zinc sulfate in a second formulation, has been demonstrated to be absorbable and physiologically active. One measure of the activity has been to assess the ability to prevent sunburns. The formulations according to the invention when properly applied to the skin have produced a biological effect equivalent to an SPF 26 sunscreen.
(b) Topical α-tocopherol has significantly ameliorated the signs and symptoms of sunburn. Topical vitamin C, as well, has been found effective in reducing sunburns.

In animal health studies, each of the vitamins has been demonstrated to prevent and reverse the signs of photo aging. Animal models have an inherent limitation and are not necessarily directly applicable to humans. For example, various mammalian species have different metabolic characteristics. In particularly, most animals have the capacity to manufacture their own vitamin C. In open clinical trials, using a similar regime, we have demonstrated the capacity of each of the vitamins to inhibit and reverse photo aging.

Open label proof of concept trials have been conducted by one of the inventors, a practicing dermatologist, and are discussed below. These trials have demonstrated the ability of a regime containing topical vitamin A (retinyl palmitate) and topical vitamin C, and α-tocopheral, combined with zinc sulfate in the preferred concentrations, to markedly reduce the frequency of actinic keratoses in human subjects with numerous lesions. Ninety-five percent of patients with actinic keratoses and malignancies exhibit sun-induced immunosuppression as a unique distinguishing characteristic. A marked reduction in frequency of new actinic keratoses in humans treated in two daily stages with the vitamin A, C and E and zinc sulfate combination according to the invention has confirmed:
(a) The effectiveness of the four basic component composition in preventing sun-induced immunosuppression.
(b) The capacity of the four basic component composition to prevent skin cancer by the reduction in frequency of actinic keratoses, and basal cell carcinoma. Patients highly prone to skin cancers, specifically basal cell carcinoma and squamous cell carcinoma, share the characteristic of sun-induced immunosuppression. Previous open label proof of concept trials conducted by a practicing dermatologist using a daily tri-vitamin composition regimen were found to reverse the existing malignancies. An alternate day regimen was found to markedly reduce the frequency of new lesions.
(c) Treatment of existing skin cancer specifically, but not limited to superficial basal carcinomas and Bowens disease, is effective in initiating reversal.
(d) The same characteristic reaction pattern of sun-induced immunosuppression is recognized to occur in 100% of patients with melanoma.

Open label proof of concept trials conducted by one of the inventors over 5 years in 300 patients initially using topical tretinoin and commercially available topical vitamin C (L ascorbic acid 17.5%) and then transitioning to the twice daily application tri-vitamin regime, demonstrated that vitamin A palmitate has a similar clinical effect to topical tretinoin with a markedly reduced frequency of irritation. Similar benefits were observed for vitamin C alone. Added topical vitamin E has been shown to enhance reversal of photo aging, notably by increasing the frequency of clinically detectable improvement in the vermillion border of the lip and reduction of fine and deep lines. This clinical trial demonstrated that the trivitamin regime and zinc sulfate has each of the above abilities to reverse photo aging. Indeed, the studies appear to indicate a synergetic effect between vitamins C and E in the morning, and vitamins A and E in the evening.

Human bodies naturally make antioxidants which protect the skin by absorbing and thereby neutralizing free radicals. Some antioxidants, however, cannot be made by the body and must be consumed daily to maintain healthy supplies. We have discovered that reasonably high levels of vitamins A, C and E applied to the skin topically in cream form, especially in a two-stage process, can increase the body's ability to absorb free radicals by a factor of up to 40 times more than normal.

The formulations according to the invention deliver vitamins and mineral zinc in a therapeutically concentrated base which enhances absorption. This maximizes the skin's ability to repair and restore itself and also to resist further sun damage.

### Specific Formulation and Application

A problem with combining vitamins A, C and E in one formulation is that because of their different physical and chemical properties, they are not necessarily compatible and effective or stable at the same pH level. In one embodiment of the invention, two cream formulations have been developed, one for daytime use and the other for nighttime use. The daytime cream contains vitamin C (L-ascorbic acid) and vitamin E (α-tocopherol) at a pH of about 3.5 and it is applied in the morning for protection during the day. In addition to the face, the cream is routinely applied to other UV exposed areas, such as the neck, the upper “V” of the chest and the back of the hands. However, the daytime cream can be safely used on any part of the body.

The nighttime cream contains vitamin A (vitamin A palmitate or retinol) and vitamin E and zinc sulfate at a pH of about 5 to 6 and is applied in the evening.

To begin treatment, it is preferable that the exposed areas of the skin are wetted with water. In the morning, a pearl size amount of the morning cream, containing vitamin C and α-tocopherol, is dispensed to the palm of one hand and rubbed together with the other hand. Then, with the palms coated, morning cream is applied gently to the face, including eyelids and lips, neck, chest and back of hands. Some people experience a slight tingling sensation lasting only for a few minutes. This usually disappears after the first week or so of treatment. Less than 1% of the population are not able to continue with the cream treatment due to side effects and allergies.

In the evening, a pearl size amount of the nighttime cream, containing vitamins A and E, and zinc sulfate, is dispensed to the palm of one hand and rubbed together with the other hand. Then, with the palms coated, nighttime cream is applied gently to the face, including eyelids and lips, neck, chest and back of hands.

In prospective open label clinical trials conducted by one of the inventors, the following benefits were noted in a predictable time course. Use of the two creams in the manner stated provides enhanced sun protection in a reasonably short period of time.

In general, the following improvements were noticed at certain stages:
6 weeks: improved smoothness and an increased overall healthy appearance of the skin.
4 months: brown patches on the skin start to fade and the skin feels pleasantly thicker and appears “dewy”.
6 months: collagen growth can be detected by feeling the enhancement of the upper lip line ridge. Fine lines are noticeably shorter and more shallow.
1 year: coarse lines, such as forehead creases, nasolabial folds, cheeks, eyes, upper lips, show marked improvement.
3-5 years: lax skin found around the eyes, jowls and neck become firm and retracted with maximum improvement of deep lines.

### Ancillary Effects

The concentrated combination of vitamins A, C and E in topical cream form has been found to be effective therapy in encouraging the body to form new collagen. Vitamin A thickens the epidermis which smooths and corrects dyspigmentation. It grows Type 1 collagen which fills in fine lines and Type VII collagen which reduces skin fragility and the frequency of skin cancer. Vitamin C corrects dyspigmentation and grows Type 3 collagen, filling in deep lines and tightening jowls. This growth of new collagen is especially helpful for several types of scarring resulting from severe acne, radiation or burns. New collagen forms under the scars, lifting and making the scars appear smooth and soft. In the same way, it lessens the appearance of stretch marks, which are a result of skin thinning during a period of rapid growth, as in pregnancy or increased physical exercise.

A common skin problem in the elderly is unsightly bruising. Chronic sun damage destroys the anchoring fibrils, which are like fine threads that connect the inner and outer layers of the skin. As a result, at the slightest pressure, the layers of skin pull apart and blood gathers between them causing a bruise to appear. We have found that vitamin A regrows these fine threads which bind the layers of skin together, thereby strengthening the skin and reducing this type of bruising.

We have invented a method of dealing with the physical and chemical incompatibility of the three vitamins and have prepared two and three-phase topical creams in which the vitamins are distributed separately. This includes both aqueous and non-aqueous phases. The vitamin C, being water soluble, can be contained in the aqueous phase while the vitamin A and E can be contained in the non-aqueous (oil) phase of a water-oil-water emulsion. Zinc sulfate, being water soluble, can be contained in the aqueous phase.

### EXAMPLE 1

### Preparation of Vitamin C&E Morning Cream by Forming Double W/O/W Emulsion

A water-oil-water (W/O/W) emulsion was produced by a two-step process. In the first step, L-ascorbic acid was dissolved in water together with magnesium sulfate and gently warmed at a temperature of about 35±5° C. The adjustment of pH to 3.5 was carried out by adding 30% sodium hydroxide solution to the L-ascorbic acid/magnesium sulfate aqueous solution. The total amount of internal aqueous phase was compensated for by adding water. Then the primary water-in-oil (W/O) emulsion was prepared by slowly adding the L-ascorbic acid/magnesium sulfate aqueous solution to the oil phase composed of hydrogenated polydecene, polyethylene glycol (30) dipolystearate and butylated hydroxytoluene at a temperature of about 70±5° C. The resulting composition was cooled with intensive stirring to a temperature of about 45±5° C. and α-tocoperol and phenonip were added to the emulsion while homogenizing the emulsion at 7000 rpm for 5 min. The primary water-oil emulsion produced was continuously stirred during cooling to room temperature.

In the second step, the primary water-oil emulsion produced in the first step was re-emulsified at 4000 rpm in an aqueous phase containing ethoxylated propylene oxide copolymer, glycerin, magnesium sulfate and phenonip with an approximately 20 g/min addition rate for the primary water-oil emulsion. After another 5 min of homogenization, the resulting water-oil-water double emulsion was stabilized sterically with the aid of 2% weight xanthan gum solution by slow addition and continuous gentle stirring until complete dispersion of the xanthan gum in solution occurred, thereby resulting in a homogeneous product formation.

The viscosity and pH of the water-oil-water product were measured as follows: Viscosity=15,000 cP±10% (Brookfield Viscometer, LVT T-E, @ 6 rpm, 25° C.) and pH=3.5. In the preparation of water-oil-water double emulsion, the lipophilic primary surfactant was a polyethylene glycol (30) dipolyhydroxystearate. The hydrophilic secondary surfactant was an ethoxylated propylene oxide copolymer. A hydrogenated polydecene and xanthan gum were used as oil and emulsion stabilizers, respectively, in the preparation of the water-oil-water emulsion.

The composition for the production of W/O/W double emulsion representing Vitamin C&E Morning Cream is summarized in Table 1.

### Preparation of Vitamin A&E Night Cream by Phase Inversion

The ingredients of Phase A (oil phase), such as emulsifying wax, caprylic/capric triglyceride and stearic acid were combined in a vessel and heated to approximately 70-75° C. with mixing until the solid components completely melted and the mixture became uniform.

The ingredients of Phase B (water phase) consisting of water, tetrasodium EDTA, panthenol, pentylene glycol and zinc sulfate were combined in a separate vessel and heated to approximately 70-75° C. with mixing until the solids completely dissolved and the mixture became uniform.

Phase B was added to Phase A whilst stirring intensively at a temperature of 70-75° C. When the batch became uniform, the heat was turned off and the agitation was switched to continuous sweeping until the mixture cooled to room temperature.

The solid and semi-solid ingredients of Phase C consisting of bulylated hydroxytoluene, butyl methoxydibenzoylmethane and retinyl palmitate were dissolved separately in a mixture of liquid components composed of α-tocopherol, bisabolol and phenonip with moderate heating to a temperature 35-40° C., and proper mixing until the mixture was uniform.

The Phase C ingredients were then added to the batch comprising the Phase A and B ingredients at a temperature of about 45° C. with homogenization for a few minutes. The emulsion produced was continuously swept during cooling to room temperature. Water loss during cream production was compensated for by adding water with sweeping mixing. The resulting product was left for equilibration overnight and remixed before filling into containers. The cream was smooth and glossy after remixing.

The viscosity and pH of the product were measured as follows: Viscosity=30,000 cP±10% (Brookfield Viscometer, LVT T-E, @ 6 rpm, 25° C.) and pH=5.5±0.5.

The compositions for the production of Vitamins A&E Night Cream is summarized in Table 1.

TABLE 1
Vitamins C&E Day Cream Formulation

| | | | |
|---|---|---|---|
| | | Manufacturer/ | Content, |
| Chemical/Trade Name | INCI Name | Supplier | % w/w |

Primary W/O emulsion (first step)
Part A

| | | | | |
|---|---|---|---|---|
| 1 | Water | Water | | QS to |
| | | | | 100 |
| 2 | Vitamin C | L-ascorbic acid | BASF | 20.00 |
| 3 | Magnesium sulfate | Magnesium sulfate | VWR Scientific | 0.70 |
| | heptahydrate | | Products | |
| 4 | Sodium hydroxide, 30% | Sodium hydroxide | Xenex Laboratories | QS to |
| | sol. | | | pH = 3.5 |

Part B

| | | | | |
|---|---|---|---|---|
| 5 | Silkflo 364-NF | Hydrogenated Polydecene | Lipo Chemicals | 15.50 |
| 6 | Arlacel P135 | PEG-30 | Uniqema | 4.00 |
| | | dipolyhydroxystearate | | |
| 7 | BHT | Butylated hydroxytoluene | Clariant | 0.20 |

Part C

| | | | | |
|---|---|---|---|---|
| 8 | Vitamin E | Tocopherol | BASF | 1.00 |
| 9 | Phenonip | Phenoxyethanol (and) | Clariant | 0.30 |
| | | Butyl Paraben (and) | | |
| | | Methyl Paraben (and) | | |
| | | Ethyl Paraben (and) | | |
| | | Propyl Paraben | | |

W/O/W Emulsion (second step)
Part D

| | | | | |
|---|---|---|---|---|
| 1 | Water | Water | | 20.10 |
| 2 | Synperonic PE/F 127 | Ethoxylated Propylene | Uniqema | 2.00 |
| | | Oxide Copolymer | | |
| 3 | Pricerine 9091 | Glycerin | Uniqema | 2.00 |
| 4 | Phenonip | Phenoxyethanol (and) | Clariant | 0.30 |
| | | Butyl Paraben (and) | | |
| | | Methyl paraben (and) | | |
| | | Ethyl paraben (and) | | |
| | | Propyl paraben | | |

Part E

| | | | | |
|---|---|---|---|---|
| 5 | Magnesium sulfate | Magnesium sulfate | VWR Scientific | 0.40 |
| | heptahydrate | | Products | |
| 6 | Keltrol, 2% sol. | Xanthan gum | Kelco Biopolymers | 0.20 |

TABLE 2
Vitamins A&E Night Cream Formulation

| | | | |
|---|---|---|---|
| | | Manufacturer/ | Content, |
| Chemical/Trade Name | INCI Name | Supplier | % w/w |

Phase A (Oil Phase)

| | | | | |
|---|---|---|---|---|
| 1 | Polawax | Emulsifying Wax | Croda Canada Ltd. | 8.00 |
| 2 | Noebee M5 | Caprylic/Capric | Stepan Company | 3.00 |
| | | Triglyceride | | |
| 3 | Stearic Acid | Stearic Acid | Croda Canada Ltd. | 0.50 |

Phase B (Water Phase)

| | | | | |
|---|---|---|---|---|
| 4 | Water | Water | | WS to |
| | | | | 100 |
| 5 | Versene 100 | Tetrasocium EDTA | Dow Chemicals | 0.10 |
| 6 | dl-Panthenol | Panthenol | Alps Pharmaceutical | 0.50 |
| 7 | Hydrolite-5 | Pentylene glycol | Symrise | 3.00 |
| 8 | Zinc sulfate heptahydrate | Zinc sulfate | Xenex Laboratories | 3.00 |

Phase C

| | | | | |
|---|---|---|---|---|
| 9 | Vitamin A palmitate | Retinyl palmitate | BASF | 1.00 |
| 10 | Vitamin E | Tocopherol | BASF | 1.50 |
| 11 | BHT | Butylated Hydroxytoluene | Clariant | 0.20 |
| 12 | Neo Heliopan 357 | Butyl methoxydibenzoyl- | Symrise | 0.50 |
| | | methane | | |
| 13 | Alpha-Bisabolol | Bisabolol | Symrise | 0.50 |
| 14 | Phenonip | Phenoxyethanol (and) | Clariant | 0.50 |
| | | Butyl paraben (and) | | |
| | | Methyl Paraben (and) | | |
| | | Ethyl paraben (and) | | |
| | | Propyl paraben | | |
| 15 | Sodium hydroxide, 30% | Sodium hydroxide | Xenex Laboratories | QS to |
| | sol. | | | pH = 5.5 ± 0.5 |

During clinical trials, it was noted that specific body areas, that is, the eyelids and lips, and specific body types characterized by blonde hair before the age of 10, were more susceptible to irritation from treatment. We discovered that the irritation in these subjects was a direct effect of the concentration of active agents applied to the skin. A second formulation for each of the day cream and night cream was created using exactly the same method of formulation but with reduced strength of the active ingredients. This markedly reduced the frequency of irritation in susceptible individuals. The composition for the reduced strength day cream is summarized as Table 3. The composition for the reduced strength night cream is summarized as Table 4. Comedogenicity was experienced by some patients and the formulation was modified to include retinol to counteract this effect. The composition for the production of Vitamins A & E & Retinol Night Cream is summarized in Table 5.

TABLE 3
Vitamins C&E Day Half Strength Cream Formulation

| | | | |
|---|---|---|---|
| | | Manufacturer/ | Content, |
| Chemical/Trade Name | INCI Name | Supplier | % w/w |

Primary W/O emulsion (first step)
Part A

| | | | | |
|---|---|---|---|---|
| 1 | Water | Water | | QS to 100 |
| 2 | Vitamin C | L-ascorbic acid | BASF | 10.00 |
| 3 | Magnesium sulfate | Magnesium sulfate | VWR Scientific | 0.70 |
| | heptahydrate | | Products | |
| 4 | Sodium hydroxide, | Sodium hydroxide | Xenex Laboratories | QS to |
| | 30% sol. | | | pH = 3.5 + 0.5 |

Part B

| | | | | |
|---|---|---|---|---|
| 5 | Silkflo 364-NF | Hydrogenated Polydecene | Lipo Chemicals | 15.50 |
| 6 | Arlacel P135 | PEG-30 | Uniqema | 4.00 |
| | | dipolyhydroxystearate | | |
| 7 | BHT | Butylated hydroxytoluene | Clariant | 0.20 |

Part C

| | | | | |
|---|---|---|---|---|
| 8 | Vitamin E | Tocopherol | BASF | 0.50 |
| 9 | Phenonip | Phenoxyethanol (and) Butyl | Clariant | 0.30 |
| | | Paraben (and) Methyl | | |
| | | Paraben (and) Ethyl Paraben | | |
| | | (and) Propyl Paraben | | |

W/O/W Emulsion (second step)
Part D

| | | | | |
|---|---|---|---|---|
| 1 | Water | Water | | 20.10 |
| 2 | Synperonic PE/F 127 | Poloxamer 407 | Uniqema | 2.00 |
| | | Oxide Copolymer | | |
| 3 | Pricerine 9091 | Glycerin | Uniqema | 2.00 |
| 4 | Phenonip | Phenoxyethanol (and) Butyl | Clariant | 0.30 |
| | | Paraben (and) Methyl | | |
| | | paraben (and) Ethyl paraben | | |
| | | (and) Propyl paraben | | |

Part E

| | | | | |
|---|---|---|---|---|
| 5 | Magnesium sulfate | Magnesium sulfate | VWR Scientific | 0.40 |
| | heptahydrate | | Products | |
| 6 | Keltrol | Xanthan gum | CPKelco | 0.20 |

The composition was prepared according to procedure for full strength Vitamins C&E Day Cream manufacturing with reduced in half concentration of Vitamins C and E.

TABLE 4
Vitamins A&E Night Half Strength Cream Formulation

| | | | |
|---|---|---|---|
| Chemical/Trade | | Manufacturer/ | Content, |
| Name | INCI Name | Supplier | % w/w |

Phase A (Oil Phase)

| | | | | |
|---|---|---|---|---|
| 1 | Polawax | Emulsifying Wax | Croda Canada Ltd. | 8.00 |
| 2 | Noebee M5 | Caprylic/Capric Triglyceride | Stepan Company | 3.00 |
| 3 | Stearic Acid | Stearic Acid | Croda Canada Ltd. | 0.50 |

Phase B (Water Phase)

| | | | | |
|---|---|---|---|---|
| 4 | Water | Water | | QS to 100 |
| 5 | Versene 100 | Tetrasocium EDTA | Dow Chemicals | 0.10 |
| 6 | dl-Panthenol | Panthenol | Alps Pharmaceutical | 0.50 |
| 7 | Hydrolite-5 | Pentylene glycol | Symrise | 3.00 |
| 8 | Zinc sulfate | Zinc sulfate | Xenex Laboratories | 3.00 |
| | heptahydrate | | | |

Phase C

| | | | | |
|---|---|---|---|---|
| 9 | Vitamin A palmitate | Retinyl palmitate | BASF | 0.50 |
| 10 | Vitamin E | Tocopherol | BASF | 0.75 |
| 11 | BHT | Butylated Hydroxytoluene | Clariant | 0.20 |
| 12 | Neo Heliopan 357 | Butyl methoxydibenzoyl- | Symrise | 0.50 |
| | | methane | | |
| 13 | Alpha-Bisabolol | Bisabolol | Symrise | 0.50 |
| 14 | Phenonip | Phenoxyethanol (and) Butyl | Clariant | 0.50 |
| | | paraben (and) Methyl | | |
| | | Paraben (and) Ethyl paraben | | |
| | | (and) Propyl paraben | | |
| 15 | Sodium hydroxide, | Sodium hydroxide | Xenex Laboratories | QS to |
| | 30% sol. | | | pH = 5.5 ± 0.5 |

The composition was prepared according to procedure for full strength Vitamins A&E Night Cream manufacturing with reduced in half concentration of vitamins A and E.

TABLE 5
Vitamins A&E&Retinol Night Full Strength Cream Formulation

| | | | |
|---|---|---|---|
| | | Manufacturer/ | Content, |
| Chemical/Trade Name | INCI Name | Supplier | % w/w |

Phase A (Oil Phase)

| | | | | |
|---|---|---|---|---|
| 1 | Polawax | Emulsifying Wax | Croda Canada Ltd. | 8.00 |
| 2 | Noebee M5 | Caprylic/Capric | Stepan Company | 3.00 |
| | | Triglyceride | | |
| 3 | Stearic Acid | Stearic Acid | Croda Canada Ltd. | 0.50 |

Phase B (Water Phase)

| | | | | |
|---|---|---|---|---|
| 4 | Water | Water | | QS to 100 |
| 5 | Versene 100 | Tetrasocium EDTA | Dow Chemicals | 0.10 |
| 6 | dl-Panthenol | Panthenol | Alps Pharmaceutical | 0.50 |
| 7 | Hydrolite-5 | Pentylene glycol | Symrise | 3.00 |
| 8 | Zinc sulfate heptahydrate | Zinc sulfate | Xenex Laboratories | 3.00 |

Phase C

| | | | | |
|---|---|---|---|---|
| 9 | Vitamin A palmitate | Retinyl palmitate | BASF | 1.00 |
| 10 | Retinol | Retinol | BASF | 1.00 |
| 11 | Vitamin B | Tocopherol | BASF | 1.50 |
| 12 | BHT | Butylated Hydroxytoluene | Clariant | 0.20 |
| 13 | Neo Heliopan 357 | Butyl methoxydibenzoyl- | Symrise | 0.50 |
| | | methane | | |
| 14 | Alpha-Bisabolol | Bisabolol | Symrise | 0.50 |
| 15 | Phenonip | Phenoxyethanol (and) | Clariant | 0.50 |
| | | Butyl paraben (and) | | |
| | | Methyl Paraben (and) | | |
| | | Ethyl paraben (and) | | |
| | | Propyl paraben | | |
| 16 | Sodium hydroxide, 30% | Sodium hydroxide | Xenex Laboratories | QS to |
| | sol. | | | pH = 5.5 ± 0.5 |

The composition was prepared according to procedure for full strength Vitamins A&E Night Cream manufacturing with 1% of retinol added. The above formulation is particularly effective in use for whitehead and blackhead prone skin.

### EXAMPLE 2

In a trial conducted by a practicing dermatologist, involving 267 patients undergoing treatment for photo ageing using slowly increasing strengths of topical tretinoin, side effects were frequent and prolonged. An average of 3.7 reactions sufficiently severe to cause discontinuation of therapy were experienced and each reaction required discontinuation of treatment for an average of 7.7 days. Only 14% of patients were able to be treated without side effects at the commercially available strength of 0.1% tretinoin. In contrast, treatment with the formulations according to the invention demonstrated that side effects occurred in only 5% of treated patients. The side effects were mild and were ameliorated by re-introducing the regime as alternate day treatment in the areas of irritation. Only 1.5% of patients were unable to tolerate the regime. In a direct comparison of this study group compared to treatment with the tri-vitamin treatment group, significantly greater efficacy with a highly significantly reduction in side effects was experienced with the formulations of the invention.

### EXAMPLE 3

An open label clinical trial of 10 patients diagnosed by a practicing dermatologist with senile purpura were treated with the tri-vitamin regime for periods of up to one year. The dermatologist reported clinically significant improvement in all patients with a reduction of the size and duration of purpura clinically detectable by 6 to 12 weeks and a marked reduction of size of post traumatic tearing by 3 months. Progressive thickening of the dermis was reported with ongoing treatment. Progressive improvement was noted for the duration of the study. In a similar manner, 3 patients with a clinical diagnosis of severe steroid atrophy were treated by a practicing dermatologist who noted a significant reduction in purpura by six weeks and tearability by 12 weeks. Clinically detectable thickening of the dermis and marked reduction of the visible signs of steroid changes were seen with continued treatment.

### EXAMPLE 4

In a study conducted by a practicing dermatologist, after informed consent, 34 volunteers were treated in the following manner. On the left side of the back, one inch squares were marked and treated daily for 3 days with 12 test compounds including a control of vehicle and a commercial identified sunscreen. After determining the MED (minimal erythema dose) for each subject, each subject was treated with one or 3 MEDs of UVB on both sides of the back. On the right side, 18 test compounds were applied including a vehicle control post sunburn at 0 hours, 8 hours, 24 and 48 hours. Measurements of the severity of the burns were recorded as the end point at 8, 24 and 48 hours.

The results demonstrated that both topically applied vitamin C and E were able to prevent the development of sunburns. The inventors found that the response was related to the concentration applied. There was a marked variability in the ability of commercial products to produce this effect. The highest strength commercial sunscreen compound produced a protection factor of approximately of 3-4.

Topical Vitamin E demonstrated a statistically visible significant treatment benefit in the treatment of sunburns. A similar review of the data of the left side of the experiment demonstrated significant visible improvement with treatment of the induced sunburns for both topical Vitamin C and E.

### EXAMPLE 5

In a trial of the same design in 10 patients comparing the described tri-vitamin formulation versus control and an SPF 30 sunscreen, the formulation according to the invention proved to be absorbable and physiologically active, and produced a SPF equivalent to a 30 sunscreen. Pre-treatment with the tri-vitamin regimen was specifically withheld in test patches for both 24 and 48 hours. It was observed that the sunburn protective effect lasted for up to 48 hours post-treatment thereby demonstrating the concept of persistent sunburn protection. In contrast, sunscreens had no persistent benefit. An increased ability to reverse sunburns versus Vitamin E alone was demonstrated.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A formulation for topical application to the skin of a human being comprising:
a first component comprising vitamin C at a concentration of about 5% to 30% weight; vitamin E at a concentration of about 1% to 10% weight; and the remainder skin compatible thickeners, emulsifiers, emollients and moisturizers, to form a mixture that has a pH of about 3.5;
a second component comprising vitamin A at a concentration of about 0.2% to 10% weight, and/or retinol at a concentration of about 0.2% to 10% weight; vitamin E at a concentration of about 1% to 10% weight: zinc sulphate at a concentration of about 2% to 5% weight, and the remainder skin compatible thickeners. emulsifiers. emollients and moisturizers, to form a mixture that has a pH of about 5.5.

2. A formulation as claimed in claim 1 wherein the vitamin A is retinyl palmitate or retinol.

3. A formulation as claimed in claim 1 wherein the vitamin C is L-ascorbic acid.

4. A formulation as claimed in claim 1 wherein the vitamin E is α-tocopherol.

5. (canceled)

6. (canceled)

7. A formulation as claimed in claim 1 wherein:
(a) the vitamin A is retinyl palmitate or retinol at a concentration of about 0.2% to 10% weight;
(b) the vitamin C is L-ascorbic acid at a concentration of about 5% to 30% weight; and
(c) the vitamin E is α-tocopherol at a concentration of about 1% to 10% weight.

8. A formulation as claimed in claim 1 wherein the first component is at least a three-phase emulsion of L-ascorbic acid in an aqueous phase and α-tocopherol in a non-aqueous phase: and the second component is a two-phase emulsion comprising sulfate in an aqueous phase and α-tocopherol and retinyl palmitate or retinol in a non-aqueous phase.

9. A formulation as claimed in claim 1 including a UV absorbing agent selected from the group consisting of butylmethoxydibenzomethane, octylmethoxycinnamate, phenylbenzimidazole sulfonic acid and 4-methylbenzylidene camphor.

10. A thee-phase formulation for topical application to the skin of a human being comprising:
(a) a water-based phase containing vitamin C at a concentration of about 5 % to 30% weight and a pH of about 3.5;
(b) an oil-based phase containing vitamin E at a concentration of about 1 % to 10% weight; the three phases being mixed and stabilized with skin compatible emulsifiers and stabilizers.

11. A formulation as claimed in claim 10 wherein the vitamin E is α-tocopherol.

12. A method of topically treating human skin to retard damage caused by exposure to sunlight comprising applying to the skin during the day a cream comprising L-ascorbic acid at a concentration of 5% to 20% weight and α-tocopherol at a concentration of 1 % to 10% weight, at a pH of 3.5; and in the evening applying to the skin a cream comprising vitamin A palmitate at a concentration of 0.2% to 10% weight and retinol at a concentration of 0.2% to 10% by weight and α-tocopherol at a concentration of 1% to 10% weight, at a pH of 5.5.

13. A method as claimed in claim 12 including at least one aqueous phase and at least one non-aqueous phase comprising L-ascorbic acid and α-tocopherol in a water-oil-water emulsion at a pH of 3.5, and vitamin A palmitate and α-tocopherol and the remainder water propanol, petrolatum and propylene glycol, in a water-oil emulsion at a pH of 5.5.

14. A method as claimed in claim 12 wherein the day cream and the night cream are used to protect against sun-induced immunosuppression, prevent sun-induced skin cancers including actinic keratoses and malignancies including squamous cell carcinomas and basal cell carcinoma, prevent photo ageing, enhance wound healing, and treat senile purpura.

15. A two-component formulation for topical application to the skin of a human being comprising:
(a) as a first component, L-ascorbic acid at a concentration of about 5% to 30% weight, α-tocopherol at a concentration of about 1 % to 10% weight, and the remainder skin compatible thickeners, emulsifiers, emollients and moisturizers, to form a cream that has a pH of about 3.5; and
(b) as a second component, retinyl palmitate or retinol at a concentration of about 0.2% to 10% weight, α-tocopherol at a concentration of about 1% to 10% weight, zinc sulfate at a concentration of about 2% to 5 % weight, and the remainder skin compatible thickeners, emulsifiers, carriers, emollients and moisturizers, to form a cream that has a pH of about 5.5.

16. A two-component formulation for topical application to the skin of a human being comprising:
(a) as a first component, vitamin C at a concentration of about 20% weight, vitamin E at a concentration of about 1 % weight, and the remainder water and skin compatible thickeners, emulsifiers, emollients and moisturizers, to form a cream that has a pH of about 3.5;
(b) as a second component, vitamin A palmitate at a concentration of about 1 % weight, vitamin E at a concentration of about 1.5% weight, zinc sulfate at a concentration of about 3 % weight, and the remainder water and skin compatible thickeners, emulsifiers, emollients and moisturizers, to form a cream that has a pH of about 5.5.

17. A formulation as claimed in claim 16 wherein the concentration of the vitamin A palinitate is about 0.5% weight, the concentration of the vitamin C is about 10% weight, the concentration of the vitamin E in the first component is about 0.5% weight and the concentration of the vitamin E in the second component is about 0.75 % weight.

18. A formulation as claimed in claim 16 including a UV absorbing agent selected from the group consisting of butyl methoxydibenzoylmethane, octyl methoxycinnamate, phenyl benzimidazole sulfonic acid and 4-methylbenzylidene camphor.
